(19) United States
(12) Patent Application Publication
(10) Pub. No.: US 2009/0227842 A1
(43) Pub. Date: Sep. 10, 2009

(54) ANGLE WIRE FIXING STRUCTURE OF ENDOSCOPE

(75) Inventor: TADASHI ANDO, Saitama-shi (JP)

Correspondence Address: BIRCH STEWART KOLASCH & BIRCH, PO BOX 747, FALLS CHURCH, VA, 22040-0747, US

(73) Assignee: FUJIFILM Corporation, Tokyo (JP)

(21) Appl. No.: 12/399,620

(22) Filed: Mar. 6, 2009

(30) Foreign Application Priority Data
Mar. 7, 2008 (JP) 2008-057146
Mar. 7, 2008 (JP) 2008-057147

(51) Int. Cl. A61B 1/005

(52) U.S. Cl. 600/146

(57) ABSTRACT

A groove is formed on both side faces of a fixing member to which a wire for bending an angle portion is fixed, a T-shaped attaching hole having a locking portion (T-shaped perpendicular portion) by which the groove for fixing is locked is provided on a side face of an angle ring, and the groove for fixing is fitted in the locking portion of the attaching hole so that the fixing member is detachably attached. On the fixing member, a screw hole may be formed so that the fixing member is screwed to a distal-end portion main body by inserting a screw through the screw hole and the attaching hole. Alternatively, on an edge of the locking portion, a perpendicular projection portion projecting in a perpendicular direction may be provided so as to enhance strength of the locking portion.

### BACKGROUND OF THE INVENTION

The disclosure of Japanese Patent Applications Nos. 2,008-057146 and 2008-057147, filed on Mar. 7, 2008, including its specification, claims and drawings, is incorporated herein by reference in its entirety.

1. Field of the Invention

The present invention relates to an angle wire fixing structure for fixing an angle wire that effects a bending operation of a distal end portion and an angle portion of an endoscope to an angle ring at a distal end side of an endoscope.

2. Description of the Related Art

The endoscope comprises a distal end portion in which an objective optical system is arranged, an angle portion (bending portion) including an angle ring in an exterior body, and an operation portion having a bending operation knob and the like, and a plurality of wires are disposed from a wire winding-up portion coupled to the bending operation knob to a distal-end ring of the angle ring. By executing a pulling operation of the plurality of wires with the bending operation knob, the distal end portion and the angle portion can be bent vertically and horizontally.

FIG. 14 illustrates a configuration of the distal end side of the conventional endoscope, and as shown in the figure, the distal end side of an endoscope 1 is constructed so that an angle ring 3 is attached on a distal-end portion main body 2. The angle ring 3 is a ring coupled body constituting a multi-nodal mechanism by pivotally attaching a large number of rings alternately in vertical and horizontal directions, and a wire 5 is attached and fixed to a attaching portion 4 provided at the distal-end ring 3. That is, the attaching portion 4 has a through hole, for example, and the wire 5 is fixed by inserting the wire 5 through the through hole and bending its distal end and then, by brazing or soldering the through hole portion.

### SUMMARY OF THE INVENTION

However, in the conventional endoscope, as described in FIG. 14, the wire 5 for the bending operation of the distal end portion and the angle portion is fixed by brazing or soldering to the angle ring 3, which causes a problem that replacement of the wire 5 is difficult. That is, in the wire 5 of the endoscope for which a diameter is to be reduced, the diameter is small and its attaching is precise, which might cause cut-off or removal at the attaching portion. In this case, in the replacement and repair of the wire 5 in FIG. 14, it is difficult to remove only the wire 5 for replacement but the entire angle portion including the angle ring 3 should be replaced at the same time, which makes the repair cumbersome. Also, since the entire angle portion is expensive, the repair cost is high, which is another problem.

Paying attention to these problems, conventional arts in the following Patent Literature 1 to 3 are proposed. Patent Literature 1 (Japanese Utility Model Laid-Open No. 3-78504) is configured such that a locking hole and a slit are provided at a locking tube arranged between a distal end portion and a distal end portion of a bending portion constituting ring, and a fixing member of a wire is inserted through the slit up to the locking hole so as to fit and lock the fixing member at the locking hole. However, with Patent Literature 1, there is a possibility that the wire fixing member is moved in an outer circumferential direction, for example, and is removed from the locking hole.

Patent Literature 2 (Japanese Patent Laid-Open No. 62-186836) has a configuration in which a groove is formed on an outer circumference of a distal-end portion main body of an endoscope, a stopper portion at a distal end of a wire is fitted in this groove, and the outside thereof is covered by a sheath tube. Since a lens assembly, a CCD and the like for an observation optical system are arranged at the distal-end portion main body in general, there is no allowance of space in the inner circumference portion, and it is difficult to fix the wire on the inner circumference side of the distal-end portion main body. Also, the distal-end portion main body is constituted by solid portions, and a hole for arranging the observation optical system, a light guide and the like is worked afterwards. Thus, if the wire is fixed on the inner circumference, a hole for the wire needs to be worked, and a structure for fixing is further made complicated. Therefore, in order to fix the wire distal end to the distal-end portion main body, a structure to fix it to the outer circumference is generally used as in Patent Literature 2.

However, since in Patent Literature 2, a groove is formed on the outer circumference of the distal-end portion main body, a thickness for the groove is needed and the diameter of the distal-end portion main body can not be reduced, and narrowing of the diameter of the endoscope is prevented. On the other hand, since the inside of the angle ring is hollow for the entirety, a space other than the space for arranging the contents such as light guide, electric cables and the like becomes a dead space, which has some allowances as compared with the distal-end portion main body, and if the wire is fixed on the angle ring side, it can be fixed on the inner circumference side and that does not enlarge the outer circumference. Thus, the wire is preferably fixed on the angle ring side.

Patent Literature 3 (Japanese Patent Laid-Open No. 59-228620) is configured such that an emboss portion using a shape memory metal is provided at a nodal ring constituting a bending portion, and a wire is attached at the time of transition of a high-temperature phase of the shape memory metal while the wire is removed at the time of transition of a low-temperature phase. However, in Patent Literature 3, since the shape memory metal is used, transition work of the high-temperature phase and the low-temperature phase is complicated, and a manufacturing costs and a repair cost are high.

The present invention was made in view of the above problems and has an object to provide an angle wire fixing structure of an endoscope in which fixing in attaching a wire is surely carried out if the wire is detachable with respect to an angle ring and moreover, the wire is not largely moved at an attaching and fixing portion in a distal-end direction, wire replacement is facilitated, a repair cost can be reduced, and narrowing of the diameter of the endoscope is not prevented.

In order to achieve the above object, an angle wire fixing structure of an endoscope according to the present invention comprises a fixing member (stopper) to which a wire for bending an angle portion is attached and in which a groove is formed on a side face thereof and an attaching hole (opening) formed on a side face of an angle ring and having a locking portion (notched portion) by which the groove of the fixing member is locked in a state where the wire is arranged on an inner circumferential face side of the angle ring, characterized in that the fixing member is detachably attached and fixed to the attaching hole.

The attaching hole is formed as a T-shaped hole with a T-shaped perpendicular portion serving as the locking portion, and the grooves formed on both side faces of the fixing member can be locked by the T-shaped perpendicular portion.

According to the above configuration, the grooves are formed on both sides of the fixing member, for example, and the T-shaped attaching hole with the T-shaped perpendicular portion serving as the locking portion is provided in the angle ring, and by inserting the fixing member from a T-shaped horizontal portion to the T-shaped perpendicular locking portion of the attaching hole and by locking the grooves on the both sides by the locking portion, the fixing member is fixed to the attaching hole.

According to the present invention as above, even if the wire is made detachable with respect to the angle ring, there is an effect that fixing in attaching of the wire can be surely carried out, and thus, wire replacement is facilitated, and the repair cost can be lowered. Also, since the wire is not attached on the distal-end portion main body of the endoscope, there is an advantage that narrowing of the diameter of the endoscope is not prevented.

In the fixing member, a screw hole is formed, and a screw can be inserted through the screw hole and the attaching hole so as to screw the fixing member into the distal-end portion main body. According to this, the fixed state can be made firmer by the screwing.

In the locking portion by which the groove of the fixing member is locked, a projection portion projecting in the perpendicular direction from the side face of the angle ring may be provided. This has an advantage that the locking portion at the attaching hole provided at the angle ring can be prevented from being widened by a frequent pulling operation of the wire.

Also, a portion of the attaching hole may be recessed on the inward side of the angle ring so that the outer circumference of the fixing member attached to the attaching hole substantially matches an outer circumferential circle of the angle ring. According to this, there are advantages that increase in the outer circumference of the attaching portion of the fixing member in the angle ring can be prevented, and the narrowing of the diameter of the endoscope is not obstructed.

Also, the angle wire fixing structure of the endoscope of another invention is provided with a wire having a small body (small mass) for locking formed at a distal end and arranged on an inner circumferential face side of an angle ring for bending an angle portion, a wire fixing portion provided for attaching a wire on the angle ring and having a locking hole for detachably locking the small body for locking formed, and a distal-end portion main body fitted and connected to the angle ring of the angle portion and having a rear end in contact with or in proximate to a distal end portion of the small body for locking, the small body for locking being arranged in a locking hole of the wire fixing portion, wherein the small body for locking is detachably attached on the locking hole of the wire fixing portion, and an operation of the small body for locking is regulated by the rear end of the distal-end portion main body.

According to the configuration of the above another invention, the wire fixing portion is integrally or detachably attached to a side face of the angle ring, the small body of the wire distal end is inserted into and locked by the locking hole (two-staged hole) of the wire fixing portion, and then, the distal-end portion main body is fitted with the angle ring and arranged so that the rear end of the distal-end portion main body is in contact with or in proximate to the distal end portion of the small body and the wire is attached and fixed to the angle ring.

The wire fixing portion may be constituted by the fixing member to which wire is attached and in which the locking hole is formed and the groove is formed on the side face and by the attaching hole formed on the side face of the angle ring and having the locking portion by which the groove of the fixing member is locked in a state where the wire is arranged on the inner circumferential face side of the angle ring, and the wire fixing portion may be constructed so that the fixing member is detachably attached to the attaching hole.

According to the above another invention, even if the wire is made detachable with respect to the angle ring, not only that the fixing at wire attachment is carried out surely but also there is an effect that the wire is not removed from the attaching and fixing portion or the wire is not elongated or cut off, either since the wire is not largely moved in the distal-end direction at the fixing portion. Therefore, the wire replacement is facilitated, and the repair cost can be reduced.

### BRIEF DESCRIPTION OF THE DRAWINGS

FIG. 1 is an exploded perspective view of a distal end portion and an angle portion illustrating an angle wire fixing structure of an endoscope according to a first embodiment of the present invention;

FIG. 2A is a front view of a fixing member in the first embodiment;

FIG. 2B is a sectional view illustrating a configuration of the distal end portion and the angle portion when a wire is attached in the first embodiment;

FIG. 3 is a diagram illustrating a variation of the fixing member and an attaching hole of the first embodiment;

FIG. 4 is an exploded perspective view of the angle portion illustrating the angle wire fixing structure of the endoscope in a second embodiment;

FIG. 5 is a sectional view illustrating a configuration of the distal end portion and the angle portion when the wire is attached in the second embodiment;

FIG. 6 is an exploded perspective view of the angle portion illustrating the angle wire fixing structure of the endoscope in a third embodiment;

FIG. 7A is a sectional view when a perpendicular projection portion in the third embodiment is fitted in a groove of the fixing member;

FIG. 7B is a sectional view illustrating a variation of the perpendicular projection portion and the groove of the fixing member in the third embodiment;

FIG. 8A is an exploded perspective view of the angle portion illustrating the angle wire fixing structure of the endoscope of a fourth embodiment;

FIG. 8B is a sectional view when the fixing member of the fourth embodiment is attached to the attaching hole;

FIG. 9A is a front view of an angle ring in the angle wire fixing structure of the endoscope according to a fifth embodiment of the present invention;

FIG. 9B is a side view of the angle ring and the wire in the fifth embodiment;

FIG. 9C is a perspective view of the distal-end portion main body in the fifth embodiment;

FIG. 10 is a sectional view illustrating the configuration of the distal end portion and the angle portion when the wire is attached in the fifth embodiment;

FIG. 11 is a sectional view illustrating a variation of the fifth embodiment;

FIG. 12 are exploded perspective views of the angle portion illustrating the angle wire fixing structure of the endoscope of a sixth embodiment;

FIG. 13 is a sectional view illustrating the configuration of the distal end portion and the angle portion when the wire is attached in the sixth embodiment; and

FIG. 14 is a sectional view illustrating a configuration of the distal end portion and the angle portion of a conventional endoscope.

### DETAILED DESCRIPTION OF THE PREFERRED EMBODIMENTS

### First Embodiment

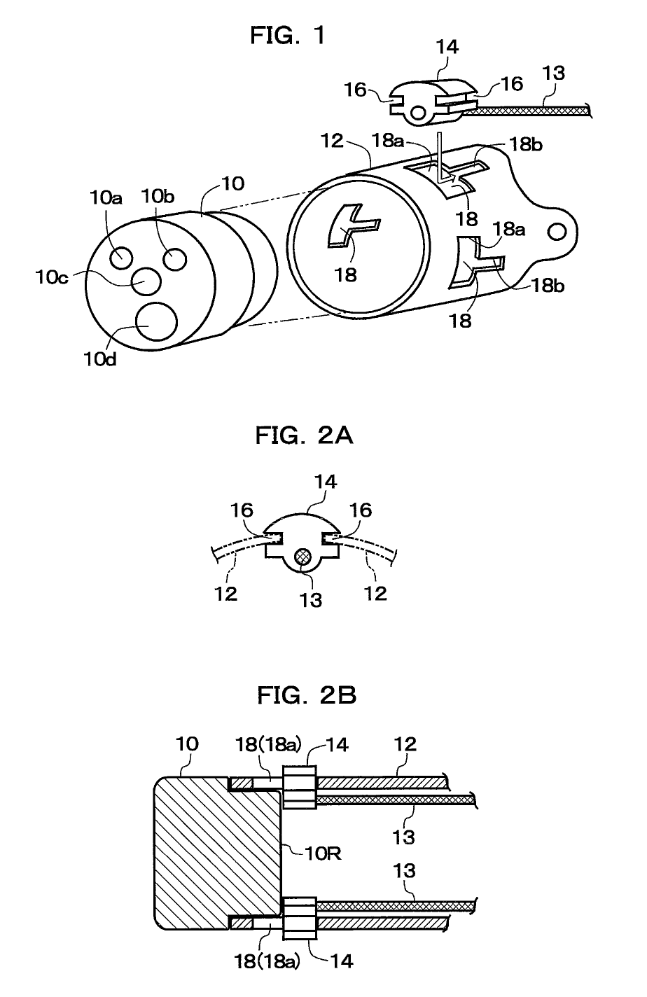

FIGS. 1 and 2 illustrate an angle wire fixing structure of an endoscope according to a first embodiment, and the endoscope comprises an insertion portion including a distal end portion and an angle portion (bending portion) and an operation portion and the like in general. In FIG. 1, a main body 10 of the distal end portion is configured to be coupled to an angle ring (the most distal end ring) 12 constituting the angle portion, and at this distal-end portion main body 10, openings 10a, 10b for an irradiation window and a light guide, an opening 10c for an observation window and an objective optical system, an opening 10d for a treatment instrument insertion channel and the like are provided.

In the first embodiment, as shown in FIG. 1 or 2A, a fixing member (stopper) 14 having a wire 13 fixed in a through hole at a lower part is provided, and the wire 13 is disposed to a winding-up wheel coupled to an angle operation knob of the operation portion. The fixing member 14 has its upper face formed in a columnar face in which grooves 16 for fixing with a width corresponding to a thickness of the angle ring 12 are formed on both side faces, and the fixing member 14 is provided at each of the four wires 13.

On the other hand, on the side face of the angle ring 12 at the most distal end, a T-shaped attaching hole (attachment/detachment hole) 18 is provided at four spots at every 90 degrees on the outer circumferential circle, and the attaching hole 18 has such a size that a T-shaped horizontal portion (front side portion) 18a can accommodate insertion of the fixing member 14, and a T-shaped perpendicular portion cut on the rear side thereof serves as a locking portion 18b by which the groove 16 for fixing is locked.

According to the first embodiment with the above configuration, in a state where the wire 13 is inserted into the angle ring 12, the fixing member 14 is inserted into the T-shaped horizontal portion 18a of the attaching hole 18 as shown in FIG. 1 and made to slide rearward while the locking portion 18b as the T-shaped perpendicular portion is fitted in the grooves 16 for fixing on the both sides (right side in the figure) so that the groove 16 for fixing is locked by the locking portion 18b, and the fixing member 14 is attached and fixed to the attaching hole 18. After that, by inserting the distal-end portion main body 10 into the angle ring 12 through the distal-end opening thereof, the state shown in FIG. 2B is realized, and the wire 13 is attached and fixed to the angle ring 12. In this case, since the fixing member 14 is not brazed or soldered to the attaching hole 18, the wire 13 is made detachable with respect to the angle ring 12, and replacement of only the wire 13 is made possible.

Moreover, in the first embodiment, since the groove 16 of the fixing member 14 is fitted in the locking portion 18b (wall body of the angle ring 12) of the attaching hole 18, a firmly fixed state can be obtained. Also, as shown in FIG. 2B, the distal-end portion main body 10 in the embodiment (length of the angle ring fitting portion) is set to a length brought into contact with the distal end face of the fixing member 14 having a rear end face 10R thereof attached to the attaching hole 18, thereby a possibility of removal of the fixing member 14 due to forward movement thereof is avoided.

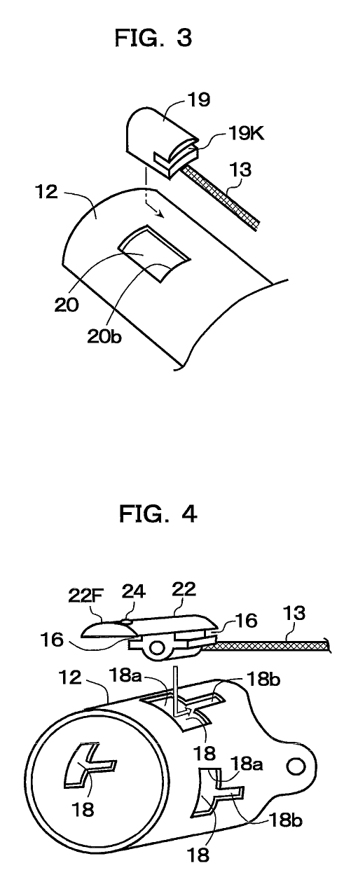

FIG. 3 shows a variation of the first embodiment, and in this variation, as shown in FIG. 3, a groove 19k for fixing is formed on the rear side of a fixing member 19 to which the wire 13 is fixed, and an attaching hole 20, which is a square hole with a size capable of accommodating insertion of the fixing member 19 and has a rear-side edge portion that serves as a locking portion 20b, is provided at the angle ring 12. With this configuration, too, by fitting the locking portion 20b of the attaching hole 20 in the groove 19k for fixing and by locking the fixing member 19 by the locking portion 20b, a firmly fixed state can be obtained.

### Second Embodiment

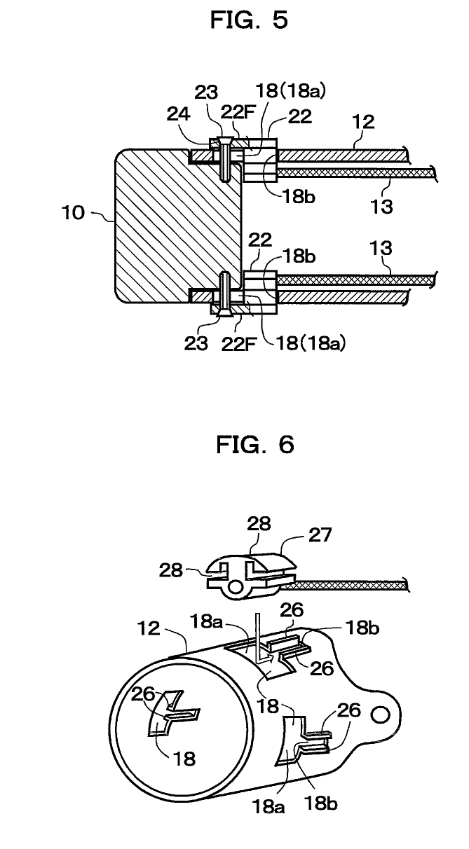

FIGS. 4 and 5 show an angle wire fixing structure of an endoscope of a second embodiment, and this second embodiment is configured to be screwed. As shown in the figure, the T-shaped attaching hole 18 having a T-shaped perpendicular portion as the locking portion 18b is provided in the angle ring 12 similarly to the first embodiment, a projection portion (a flange portion) 22F is integrally formed on a fixing member 22 having the groove 16 formed so as to protrude to the front side above it, and a screw hole 24 through which a screw 23 is inserted is formed on the projection portion 22F.

According to the configuration of the second embodiment, as shown in FIG. 5, in a state where the locking portion 18b is fitted in the groove 16 of the fixing member 14, the fixing member 22 is attached to the attaching hole 18, and the screw 23 can be screwed and fixed to the distal-end portion main body 10 through the screw hole 24 and the attaching hole 18 (18a). Therefore, in the second embodiment, not only that the fixing member 22 is fixed by the locking between the groove 16 thereof and the locking portion 18b but also the member is screwed, and the fixing member 22 and hence the wire 13 are firmly fixed. On the other hand, replacement of only the wire 13 is made possible by removing the screw 23.

### Third Embodiment

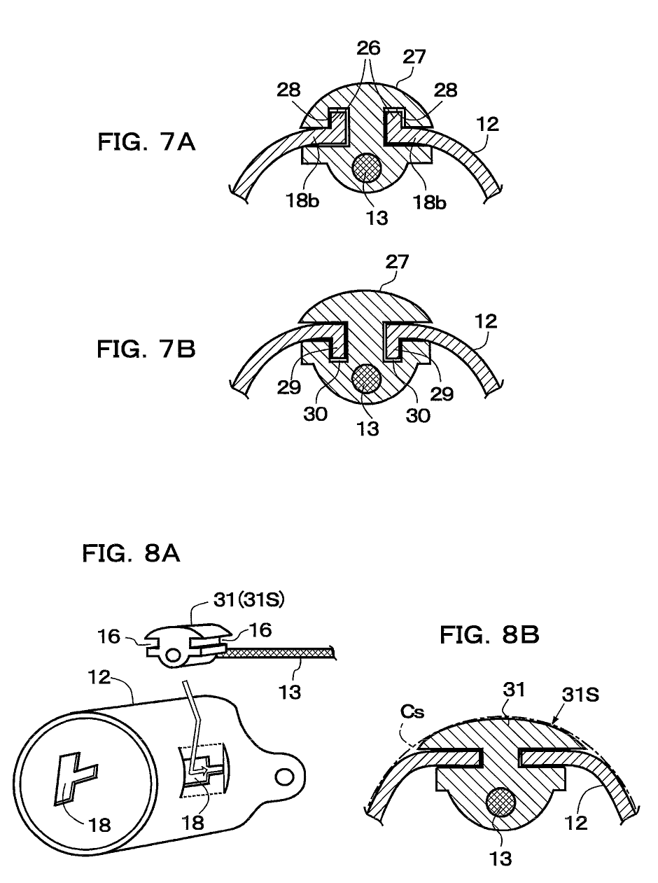

FIGS. 6 and 7 show an angle wire fixing structure of an endoscope of a third embodiment, and in the third embodiment, a perpendicular projection portion is provided at a locking and fixing portion of an attaching hole. As shown in FIG. 6, the attaching hole 18 similar to that in the first embodiment is provided at the angle ring 12 in the third embodiment, but in an edge of the locking portion (T-shaped perpendicular portion) 18b (longitudinal direction) of the attaching hole 18, a perpendicular projection portion 26 having an edge portion thereof folded outward in the perpendicular direction is formed. On the other hand, in a fixing member 27, as also shown in FIG. 7A, a groove 28 for fixing with an L-shaped section is formed.

According to the configuration of the third embodiment, as shown in FIG. 7A, by fitting the L-shaped locking portion 18b including the perpendicular projection portion 26 in the L-shaped groove 28 of the fixing member 27, the wire 13 (distal-end fixing member 27) can be detachably attached to the angle ring 12 (attaching hole 18). Moreover, since the perpendicular projection portion 26 is provided in the L-shape, strength of the locking portion 18b itself is enhanced, and even in the frequent pulling operation of the wire 13, the locking portion 18b would not be widened open.

FIG. 7B shows a variation of the third embodiment, and as shown in the figure, in this variation, a perpendicular projection portion 29 having an edge portion thereof folded inward in the perpendicular direction is provided at the locking portion 18b of the attaching hole 18, and a groove 30 for fixing in an inverted L-shape is formed in the fixing member 27. With this configuration, too, the same effect as above can be obtained.

### Fourth Embodiment

FIG. 8 shows an angle wire fixing structure of an endoscope of a fourth embodiment, and the fourth embodiment is configured so that an outer circumference of the fixing member complies with an outer circumferential circle of the angle ring. As shown in FIG. 8, in the fourth embodiment, a portion of the attaching hole 18 similar to that in the first embodiment (portion including the periphery) is recessed inward and formed into a flat face, and a sectional outer circumferential line (outer circumferential curved face) 31S of a fixing member 31 is formed so as to become the same (radius) as a sectional outer circumferential circle (outer circumferential curved face) of the angle ring 12.

According to the fourth embodiment, as shown in FIG. 8B, the outer circumferential line 31S of the fixing member 31 matches an outer circumferential circle Cₛ of the angle ring 12, the fixing member 31 does not protrude from the outer circumference of the angle ring 12, and thus, there is an advantage that the wire 13 is made detachable and a firm fixed state can be obtained without preventing narrowing of the diameter of the angle portion (insertion portion) and hence, the endoscope.

### Fifth Embodiment

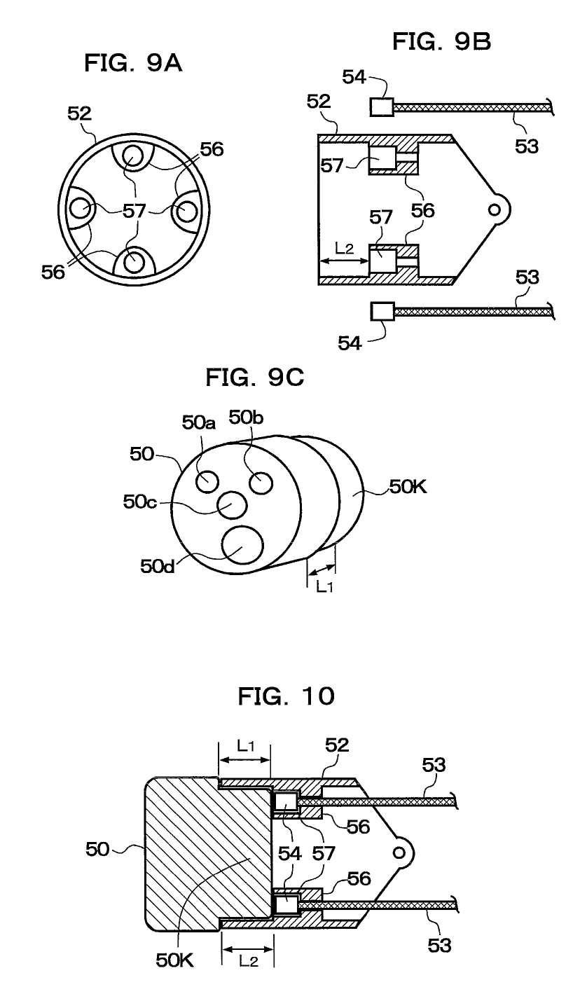

FIGS. 9 and 10 show an angle wire fixing structure of an endoscope according to a fifth embodiment, and the endoscope comprises an insertion portion including a distal end portion and an angle portion (bending portion) and an operation portion and the like in general. That is, a main body 50 at the distal end portion in FIG. 9C is configured to be coupled to an angle ring (most distal end ring) 52 in FIG. 9B constituting the angle portion, and the distal-end portion main body 50 has openings 50a, 50b for an irradiation window and a light guide, an opening 50c for an observation window and an objective optical system, and an opening 50d for a treatment instrument insertion channel and the like.

In the fifth embodiment, as shown in FIG. 9, a cylindrical small body (stopper) 54 for locking is fixed to a distal end of a wire 53 by brazing, soldering and the like, and the wire 53 is disposed up to a winding-up wheel coupled to an angle operation knob of the operation portion. The small body 54 may be a small mass obtained by folding and brazing the wire distal end. On the other hand, on the inner circumference of the angle ring 52 at the most distal end, wire fixing portions 56 are provided at four spots at every 90 each (they are depicted in a larger way than they actually are), for example, and in the wire fixing portion 56, a locking hole (through hole) 57 constituted by a small-diameter rear-side tube portion on which the wire 53 is arranged and a large-diameter front-side tube portion communicating with the tube portion and provided with a step for locking the small body 54 is formed. A length of the front-side tube portion (length in the axial direction) of the locking hole 57 is formed substantially the same as the length of the small body 54.

A length L₁ [FIG. 9C] of a fitting portion 50K into an angle ring on the rear side of the distal-end portion main body 50 is set substantially the same length as a length L₂ [FIG. 9B] from the distal end of the angle ring 52 to the wire fixing portion 56 (L₁≈L₂), by which movement (position) of the small body 54 can be regulated by the rear end of the distal-end portion main body 50.

According to the fifth embodiment with the above configuration, as shown in FIG. 10, the wire 53 is inserted through the locking hole 57 of the wire fixing portion 56 in the angle ring 52 and arranged, the small body 54 at the distal end of the wire 53 is locked by the locking hole 57, and then, the distal-end portion main body 50 is fitted through the distal-end opening of the angle ring 52 so as to attach the wire 53. At this time, the rear end of the distal-end portion main body 50 (fitting portion 50K) is brought into contact with the distal end portion of the small body 54 and holds the small body 54 so that the movement of the small body 54 in the distal-end direction is regulated.

As mentioned above in the fifth embodiment, since the small body 54 is not brazed or soldered to the locking hole 57 of the fixing portion 56, by removing the distal-end portion main body 50 from the angle ring 52, the wire 53 is made detachable with respect to the angle ring 52, and replacement of only the wire 53 is made possible. Also, since the small body 54 is held by the rear end of the fitting portion 50K (distal-end portion main body 50) having substantially the same length as the length from the distal end of the angle ring 52 to the wire fixing portion 56 (L₁≈L₂), removal of the wire 53 from the fixing portion 56 or elongation or cut-off in the wire 53 is not caused.

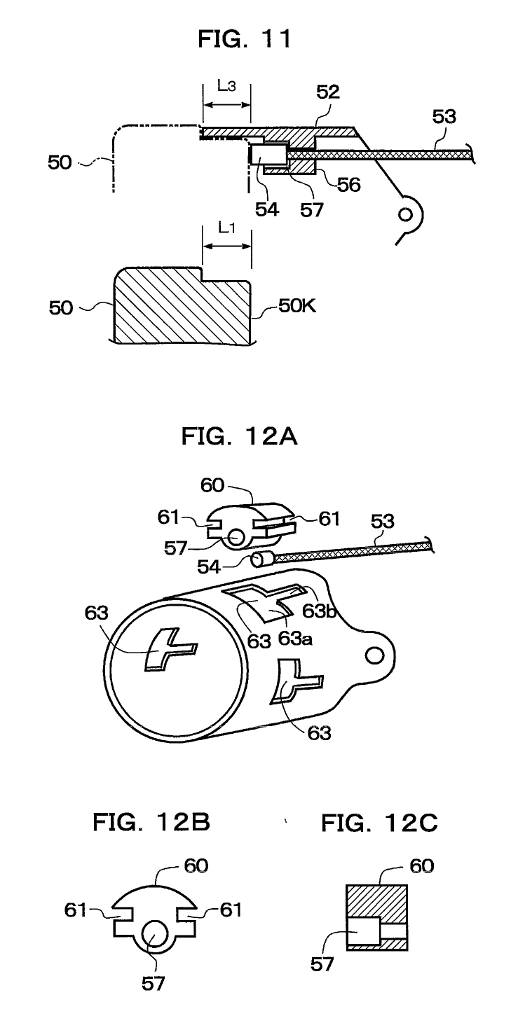

FIG. 11 shows a variation of the fifth embodiment, in which the length of the small body 54 is longer than the depth of the locking hole 57. In this case, by setting a length L₃ from the distal end of the angle ring 52 to the distal end of the small body 54 substantially the same as a length L₁ of the fitting portion 50K to the angle ring of the distal-end portion main body 50 (L₁≈L₃), the movement of the small body 54 in the distal-end direction can be regulated.

### Sixth Embodiment

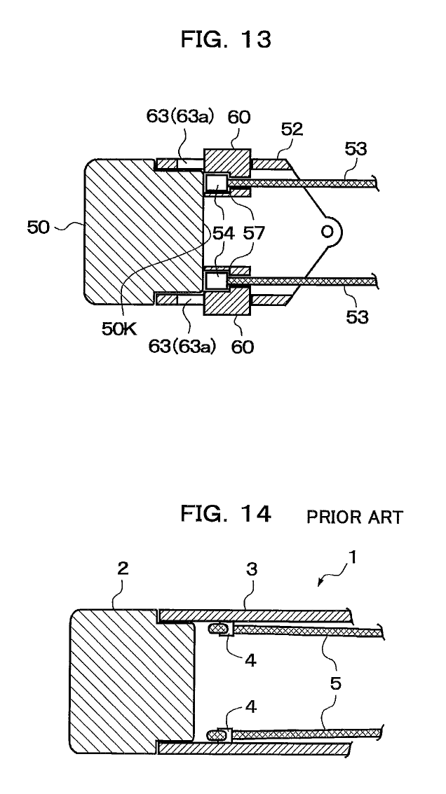

FIGS. 12 and 13 show an angle wire fixing structure of an endoscope of a sixth embodiment, and in the sixth embodiment, the wire fixing portion is made detachable. As shown in the figure, in the sixth embodiment, as shown in FIG. 12, similarly to the above embodiments, a fixing member 60 having the locking hole 57 formed in which a rear-side tube portion on which the wire 53 is arranged and a front-side tube portion provided with a step for locking the small body 54 is provided, and the small body 54 of the wire 53 is locked by the locking hole 57 of the fixing member 60. The fixing member 60 has its upper face as a columnar face and has a groove 61 for fixing formed with a width corresponding to the thickness of the angle ring 52 on both side faces and arranged at each of the four wires 53.

On the other hand, on the side face of the angle ring 52 at the most distal end, a T-shaped attaching hole (attachment/detachment hole) 63 is provided at four spots at every 90 degrees on the outer circumferential circle, and in the attaching hole 63, a T-shaped horizontal portion (front side portion) 63a is of the size through which the fixing member 60 can be inserted, and a T-shaped perpendicular portion cut-in on the rear side serves as a locking portion 63b for locking the groove 61 for fixing. The configurations of the distal-end portion main body 50, the lengths L₁, L₂ of the angle ring 52 and the like are the same as those in the fifth embodiment.

According to the sixth embodiment with the above configuration, as shown in FIG. 13, in a state where the small body 54 of the wire 53 is locked by the locking hole 57 of the fixing member 60, the fixing member 60 is inserted into the T-shaped horizontal portion 63a of the attaching hole 63, and the locking portion 63b as the T-shaped perpendicular portion is made to slide rearward (right side in the figure) while being fitted in the groove 61 on the both sides so that the groove 61 for fixing is locked by the locking portion 63b and the fixing member 60 is attached and fixed to the attaching hole 63. After that, by inserting the distal-end portion main body 50 into the angle ring 52 through the distal-end opening, the wire 53 is attached and fixed to the angle ring 52.

In this case, too, by removing the distal-end portion main body 50 from the angle ring 52, the wire 53 is made detachable with respect to the angle ring 52, and replacement of only the wire 53 is made possible. Also, since the movement of the small body 54 is regulated by the rear end of the distal-end portion main body 50, removal, elongation or cut-off of the wire 53 is not caused. Moreover, there is an advantage that, even in the case where the fixing member 60 is broken, repair is possible by replacing only the fixing member 60.

Description of Symbols

| | |
|---|---|
| 2, 10, 50 | distal-end portion main body |
| 3, 12, 52 | angle ring |
| 5, 13, 53 | wire |
| 14, 19, 22, 27, 31, 60 | fixing member |
| 16, 28, 30, 61 | groove |
| 18, 20, 63 | attaching hole |
| 23 | screw |
| 24 | screw hole |
| 26, 29 | perpendicular projection portion |
| 31S | sectional outer circumferential line |
| 50K, 56 | fitting portion |
| 54 | small body |
| 57 | locking hole |

What is claimed is:

1. An angle wire fixing structure of an endoscope, comprising:
a fixing member to which a wire for bending an angle portion is attached and in which a groove is formed on a side face thereof; and
an attaching hole formed on a side face of an angle ring and having a locking portion by which the groove of the fixing member is locked in a state where the wire is arranged on an inner circumferential face side of the angle ring, wherein
the fixing member is detachably attached and fixed to the attaching hole.

2. The angle wire fixing structure of an endoscope according to claim 1, wherein the attaching hole is formed as a T-shaped hole with a T-shaped perpendicular portion serving as the locking portion, and the groove formed on both side faces of the fixing member is locked by the T-shaped perpendicular portion.

3. The angle wire fixing structure of an endoscope according to claim 1, wherein a screw hole is formed in the fixing member, and a screw is inserted through the screw hole and the attaching hole so as to screw the fixing member to a distal-end portion main body.

4. The angle wire fixing structure of an endoscope according to claim 1, wherein a projection portion projecting in a perpendicular direction from the angle ring side face is provided in the locking portion by which the groove of the fixing member is locked.

5. The angle wire fixing structure of an endoscope according to claim 1, wherein a portion of the attaching hole is recessed on the inward side of the angle ring so that an outer circumference of the fixing member attached to the attaching hole substantially matches an outer circumferential circle of the angle ring.

6. An angle wire fixing structure of an endoscope, comprising:
a wire having a small body for locking formed at a distal end and arranged on an inner circumferential face side of an angle ring for bending an angle portion;
a wire fixing portion provided for attaching the wire to the angle ring and having a locking hole formed for detachably locking the small body for locking; and
a distal-end portion main body fitted and connected to the angle ring of the angle portion and having a rear end in contact with or in proximate to a distal end portion of the small body for locking, the small body for locking being arranged in the locking hole of the wire fixing portion, wherein
the small body for locking is detachably attached to the locking hole of the wire fixing portion, and movement of the small body for locking is regulated by the rear end of the distal-end portion main body.

7. The angle wire fixing structure of an endoscope according to claim 6, wherein the wire fixing portion is integrally or detachably attached to a side face of the angle ring.

8. The angle wire fixing structure of an endoscope according to claim 6, wherein the wire fixing portion is constituted by a fixing member to which a wire is attached and in which the locking hole is formed and a groove is also formed on a side face and an attaching hole formed on a side face of the angle ring and having a locking portion by which the groove of the fixing member is locked in a state where the wire is arranged on an inner circumferential face side of the angle ring; and
the fixing member is constructed to be detachably attached to the attaching hole.
